(19) United States
(12) Patent Application Publication
(10) Pub. No.: US 2018/0244761 A1
(43) Pub. Date: Aug. 30, 2018

(54) NOVEL ANTIBODY AGAINST RIBOSOMAL PROTEIN S3 AND COMPOSITION FOR DIAGNOSING BIOLOGICAL STRESS

(71) Applicant: Joon KIM, Seoul (KR)

(72) Inventors: Joon KIM, Seoul (KR); Hag Dong KIM, Gyeonggi-do (KR); Youjin JUNG, Seoul (KR)

(21) Appl. No.: 15/666,301

(22) Filed: Aug. 1, 2017

(30) Foreign Application Priority Data
Feb. 28, 2017 (KR) 10-2017-0026571

(51) Int. Cl. C07K 16/18; C07K 14/47; G01N 33/68

(52) U.S. Cl. CPC C07K 16/18; C07K 14/47; C07K 2319/40; C07K 2317/34; G01N 33/6893

(57) ABSTRACT

The present invention relates to an antibody acting as a stress sensor by sensing various stresses in a living organism including a human. Particularly, the present invention relates to a novel antibody specifically recognizing the above protein modifications, and a composition for diagnosing a biological stress including the antibody, based on the fact that one molecular ubiquitin protein binds to lysine residue of ribosomal protein S3 (rpS3) in a stressed state so that the ubiquitin protein is modified. In addition, the present invention relates to a simple and accurate kit for diagnosing the biological stress using the same.

### CROSS-REFERENCE TO RELATED APPLICATION(S)

The present application claims priority to Korean Patent Application No. 10-2017-0026571, filed on Feb. 28, 2017, which is herein incorporated by reference in its entirety.

### BACKGROUND OF THE INVENTION

### 1. Field of the Invention

The present invention relates to an antibody acting as a stress sensor by sensing various stresses in a living organism including a human. Particularly, the present invention relates to a novel antibody and a composition comprising the antibody suitable for specifically recognizing protein modifications due to various stresses and, in particular, for diagnosing biological stress based on the fact that one molecular ubiquitin protein binds to lysine residue of ribosomal protein S3 (rpS3) in a stressed state so that the ubiquitin protein is modified. In addition, the present invention relates to a simple and accurate kit for diagnosing the biological stress using the same.

### 2. Description of the Related Art

A ribosome is an organelle which biosynthesizes all proteins in a living organism, and consumes most energy. Although it is not well known with respect to a human cell, the ribosome for a microorganism is an important organ enough to consume about 80% of biological energy when an individual grows. A living organism may stop protein synthesis and use the saved energy to overcome a stress. Ribosomal protein S3 (rpS3) is known as a component of the ribosome, and known as enzyme for detecting and repairing DNA damage. Recently, in addition to the above functions, it has been found that the rpS3 has various additional extra-ribosomal functions in response to the biological stress, for example, a function related to DNA repair, cancer metastasis, apoptosis, and transcription in an immune response. Recently, it has been reported that the physiological activity of the rpS3 protein is regulated by ubiquitin which is a protein consisting of 76 to 121 amino acids, or ubiquitin-like proteins such as a neural precursor cell expressed, developmentally down-regulated-8 (NEDD8) and a small ubiquitin-related modifier-1 (SUMO-1) (Herhaus et al., 2015 EMBO Rep. 16(9): 1071-83).

It was reported that the rpS3 protein is modified by the NEDD8 in nucleolus of a cell line treated with actinomycin D or MG132 which are protein synthesis inhibitors, and the neddylation may be associated with degradation at the nucleus of the rpS3 protein (Xirodimas et al., 2008, EMBO Rep. 9(3): 280-286).

It is known that the rpS3 protein is protein-modified by the SUMO-1 at a residue of Lys18, Lys214 or Lys230, and the sumoylation increases the stability of the rpS3 protein (Jang et al., 2011, Biochem Biophys Res Commun. 414(3): 523-7).

The ubiquitylation of the rpS3 protein may be classified into two cases. The first case of ubiquitination is polyubiquitination. The polyubiquitination of the rpS3 protein induces proteasome-dependent degradation, which occurs when intracellular heat-shock protein 90 (Hsp90) protein is blocked from binding to the rpS3 protein. The degradation of the rpS3 protein is an important mechanism for intracellular retention of ribosomal components (Kim et al., 2006, Mol Biol Cell. 17(2): 824-33). In contrast, the monoubiquitination has entirely different function.

The second case of ubiquitination is monoubiquitination. When the living organism is stressed, human rpS3 protein is mono-ubiquitylated. Because lysine 214 residue of the protein is protein-modified, and the modification stops an elongation step among protein biosynthesis steps of the ribosome, the modification is very important to stop the protein synthesis. When the lysine 214 residue of the rpS3 protein is prevented from being mono-ubiquitylated by mutating the lysine 214 residue, the protein synthesis does not stop, thereby inducing the generation of wrong protein biosynthesis in the ribosome, thus apoptosis is stimulated. Accordingly, the mono-ubiquitylation of the intracellular rpS3 protein is an important mechanism for maintaining the homeostasis of the ribosome (Higgins et al., 2015 Mol. Cell. 59(1), 35-49).

### SUMMARY OF THE INVENTION

The present invention relates to an antibody acting as a stress sensor by sensing various stresses in a living organism. Particularly, one ubiquitin protein binds to lysine residue of ribosomal protein S3 [rpS3 (human) and Rps3p (yeast)] under stressed conditions and protein modification occurs, thus the protein synthesis of cells stops. The antibody may be used as a measuring indicator for the biological stress by specifically sensing the modification of the above protein.

The inventors have completed the present invention by preparing a polyclonal antibody specifically recognizing the sequence of a specific type of amino acid in which the ubiquitin protein binds to the lysine residue 214 of the human rpS3 protein, and by proving that the antibody specifically recognizes the rpS3 protein with the bound ubiquitin at the 214 residue of human rpS3 protein.

Accordingly, an object of the present invention is to provide a polyclonal antibody which specifically recognizes a specific form of the ubiquitin binding to the lysine residue 214 of the rpS3 protein, and a composition for diagnosing a biological stress including the antibody.

In the present invention, in order to prepare an antibody which specifically recognizes a specific form of one ubiquitin binding to the lysine residue 214 of the rpS3 protein, peptides from the lysine residue 214 to a residue 218 of the human rpS3 protein (Genbank EAW74963.1) were synthesized as you can see from FIGS. 1a and 8a and L-shape peptide binding to six amino acids from glycine at a carboxyl terminal of the ubiquitin protein through isopeptide-binding to the epsilon amino group of the lysine 214 of the rpS3 protein is used as an immunogen as you can see from FIG. 1a and dotted box area of 8b. The reason to synthesize L-shape peptide is to raise antibodies which are recognizing residues after 214 and ubiquitin together. This area is specific for a human protein.

The present invention relates to an antibody which specifically recognizes mono-ubiquitinated (monoU) rpS3, in which the antibody can be used as an indicator for simply and accurately measuring the degree of biological stress of various eukaryotes from yeast to a human. Particularly, the yeast is widely used to produce bread, beer and so on, and the yield decreases when cells are stressed, thus it can be useful for a suitable culture indicator for the stress, so that the yeast can be very useful industrially.

### BRIEF DESCRIPTION OF THE DRAWINGS

FIG. 1a shows L-shape peptide in which ubiquitin protein binds to lysine residue 214 of human rpS3 protein.

FIG. 1b shows T-shape peptide in which ubiquitin protein binds to lysine residue 214 of human rpS3 protein.

FIG. 1c shows a method to purify antibody specific for monoubiquitinated Rps3 protein.

FIG. 2 shows a plasmid gene map of human rpS3 whole protein coding gene for expressing GST-rpS3 protein binding to Affi-Gel 10 resin so as to the affinity purification and refinement for a produced polyclonal antibody against L or T form antigen described FIGS. 1a and b.

FIG. 3a shows detection patterns of mono-ubiquitinated rpS3 when human HT1080 cell line is subject to ultraviolet rays.

FIG. 3b shows mono-ubiquitinated rpS3 detected upon irradiation with ultraviolet rays, after plasmid obtained by substituting rpS3 214 lysine with arginine is put into human HT1080 cell line.

FIG. 4a shows a plasmid gene map of yeast Rps3 whole protein coding gene expression plasmid used for an immunizing antigen.

FIG. 4b shows a plasmid gene map of yeast Rps3 whole protein coding gene expression for expressing GST-Rps3 protein binding to Affi-Gel 10 resin so as to the affinity purification and refinement for a produced polyclonal antibody.

FIG. 5 shows a Western blotting of Rps3 proteins from human cells, Candida, and yeast using a yeast polyclonal antibody, respectively.

FIG. 6 shows detection patterns of mono-ubiquitinated Rps3 when yeast is subject to 3-Amino-1,2,4-triazole (3-AT) (A), rapamycin (B) or ultraviolet rays (C).

FIG. 7 shows mono-ubiquitinated Rps3 detected upon irradiation with ultraviolet rays in a strain obtained by substituting yeast Rps3 212 lysine with arginine.

FIG. 8a shows the comparison of amino acid sequences of Rps3 proteins from a human, Candida albicans (Ca) and yeast Saccharomyces cerevisiae (Sc). The boxed area with solid lines or dotted lines indicates the Rps3 region for L form or T form immunogen respectively.

FIG. 8b square box shows the comparison of amino acid sequences of ubiquitin proteins from a human, Candida albicans (Ca) and yeast Saccharomyces cerevisiae (Sc). The long boxed area with solid lines or dotted lineindicates the ubiquitin region for L form or T form immunogen respectively.

### DETAILED DESCRIPTION OF THE INVENTION

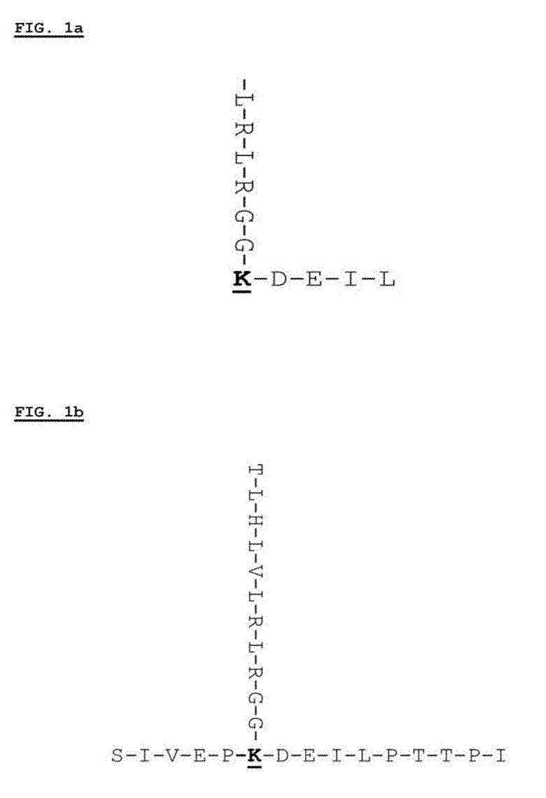

The present invention includes the steps of: (a) synthesizing peptides (K-D-E-I-L) from the lysine residue 214 to the residue 218 of human rpS3 protein; and (b) binding six residues (G-G-R-L-R-L) located at the carboxyl terminal of the ubiquitin protein to the epsilon-amino group (ε-amino group) of lysine 214 of the peptides, and includes a method of preparing the peptide of FIG. 1.

In addition, the present invention includes a method of preparing an antibody using the peptide prepared by the above method as an antigen.

The present invention includes an antibody prepared by using a protein having an amino acid structure of FIG. 1a as an antigen.

The antibody specifically binds to the rpS3 protein binding to the ubiquitin protein, thus the antibody may be used to diagnose biological stress.

The present invention includes a composition for diagnosing a biological stress, which includes an antibody which specifically binds to the rpS3 protein binding to the ubiquitin protein. The antibody may be a monoclonal or polyclonal antibody.

In addition, the present invention provides a kit for diagnosing a biological stress including the above antibody composition.

Hereinafter, the present invention will be described in more detail with reference to examples. Although the examples are provided for further comprehension of the present invention, the scope of the present invention is not limited thereto.

### Example 1

Preparation of Antigen

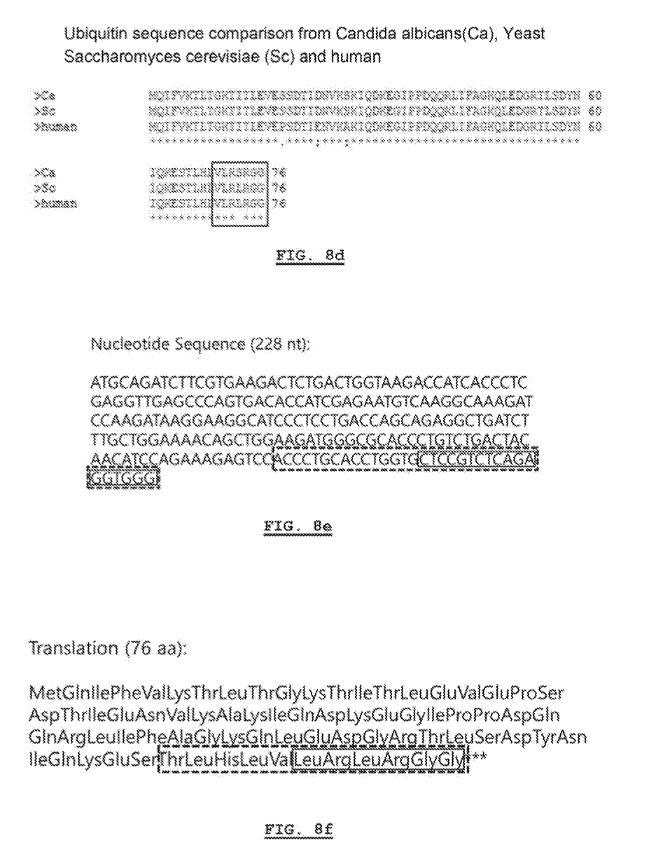

Peptides (K-D-E-I-L) were synthesized from the lysine residue 214 to the residue 218 of the human rpS3 protein (Genbank EAW74963.1). Then, last six residues of ubiquitin (G-G-R-L-R-L) (FIG. 1a and boxed area with solid lines in FIG. 8b) including glycine positioned at the carboxyl terminal of the ubiquitin protein were isopeptide-bonded to the epsilon-amino group (ε-amino group) of the lysine 214 of the rpS3 protein. (FIG. 8)

Finally, the L-shape peptide shown in FIG. 1a was prepared as an immunogen. The L-shape peptide is based on a T-shape polypeptid in which the ubiquitin binds to the lysine residue 214 of the rpS3 protein actually present in the cell (FIG. 1b). L-shape peptide (boxed area with solid lines of FIGS. 8a and 8b) was made first because this peptide is shorter than T-shape peptide. It was synthesized from the C-terminal L amino acid to N-terminal K residue and then isopeptidation was performed at the reside 214 lysine and glycine residue of ubiquitin. To make T-shape peptide shown in FIG. 1b, peptides from the serine residue 209 to the isoleucine residue 223 (boxed area with dotted lines of FIG. 8b) which are derived from human rpS3 protein was synthesized from the C-terminal 223 isoleucine residue to the N-terminal 209 serine residue was synthesized and glycine residue of ubiquitin was attached by isopeptidation as you can see in FIG. 1b and FIG. 8.

### Example 2

Immunity

The peptide (antigen) prepared in Example 1 was intravenous-injected into a rabbit about 20 weeks old. The immunity was reinforced through the second inoculation after two weeks from the first inoculation, and then the third inoculation after three weeks from the second inoculation.

### Example 3

Purification of Polyclonal Mono-Ubiquitin Antibody

In order to purify polyclonal antibodies in plasma collected from the rabbit completed with the third immunization in Example 2 by using a humanitarian manner, the example was carried out as follows:

First, dissolve 5 mg each of either L or T form peptide in PBS buffered saline at 4° [symbol] . Then react the dissolved buffer with 1 mL of Affi-Gel 10 resin(Bio-Rad, Cat. #1536099) so that L or T form peptide is fixed to the resin See first stage of FIG. 1C.

Then, the reacted resin was incubated with plasma collected from the immunized mouse overnight at pH 7.5. See second stage of FIG. 1C. Then an antibody elution buffer (0.1M Citric acid pH3 4° C.) was applied, thus the antibodies specifically binding to the L or T form peptide were isolated. See third and fourth stages of FIG. 1C. After completion of the elution, the buffer was neutralized with 1M Tris pH 9.0 buffer. This eluent contains antibodies which specifically bind to each peptide. (FIG. 1c)

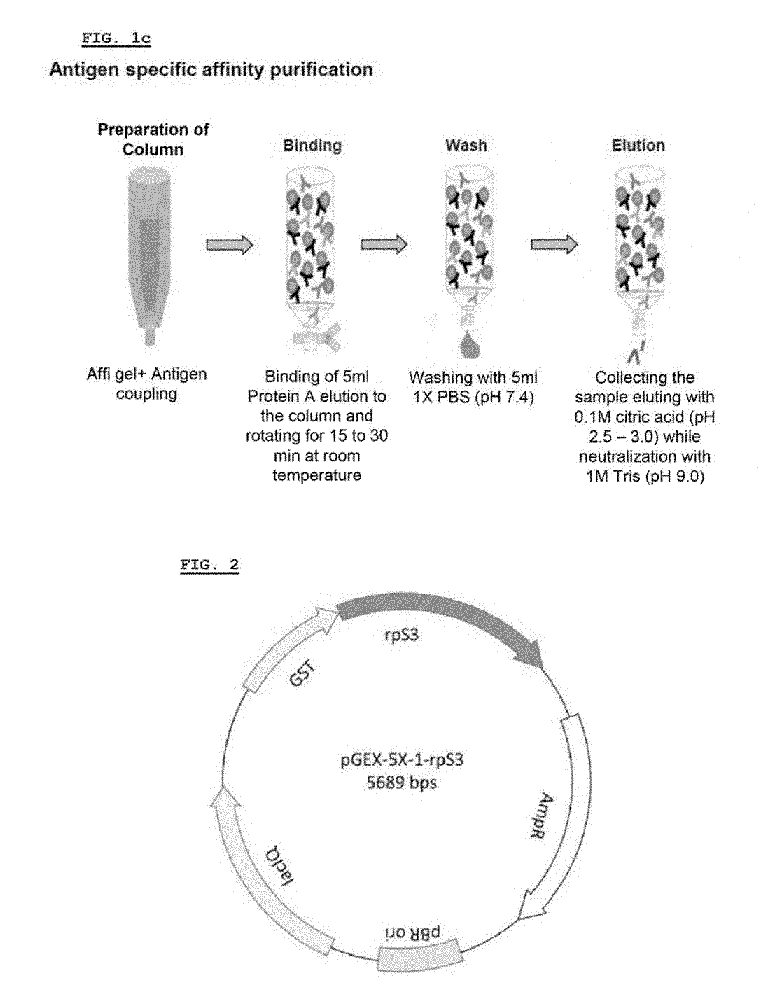

In FIG. 2

### Example 4

Western Blotting Experiment on Human Cells by Using Mono-Ubiquitin Antibody

In order to confirm whether the polyclonal antibody obtained in Example 3 specifically recognizes the form of ubiquitin binding to lysine 214 of the rpS3 protein, a Western blotting experiment was performed.

First, a human cell line HT1080 cells were irradiated with ultraviolet rays (UV 150 J/m²) as a stress source and harvested according to each time, and each cell was put in Tris-NaCl-sodium deoxycholate buffer (TND buffer) containing protease inhibitors, and then lysed. The above obtained cell lysate was quantitated, the Western blotting experiment was performed by using two kinds of antibodies with respect to 5 μg of each protein.

First human antibody is for recognizing whole rpS3 protein, the following processes were performed to prepare the first human antibody. First, the rpS3 protein was labeled with 6xHis and overexpressed in E. coli (as described previously in Korean patent application No. 10-2017-0069289) and which is incorporated herein by reference for all purposes, and the protein was isolated and purified using nickel-nitrilotriacetate (NTA) agarose resin. The isolated 6xHis-rpS3 fusion protein was intravenous-injected into a rabbit about 20 weeks old, thereby preparing polyclonal antibodies.

The second antibody is the mono-ubiquitinated rpS3-specific polyclonal antibody obtained in Example 3.

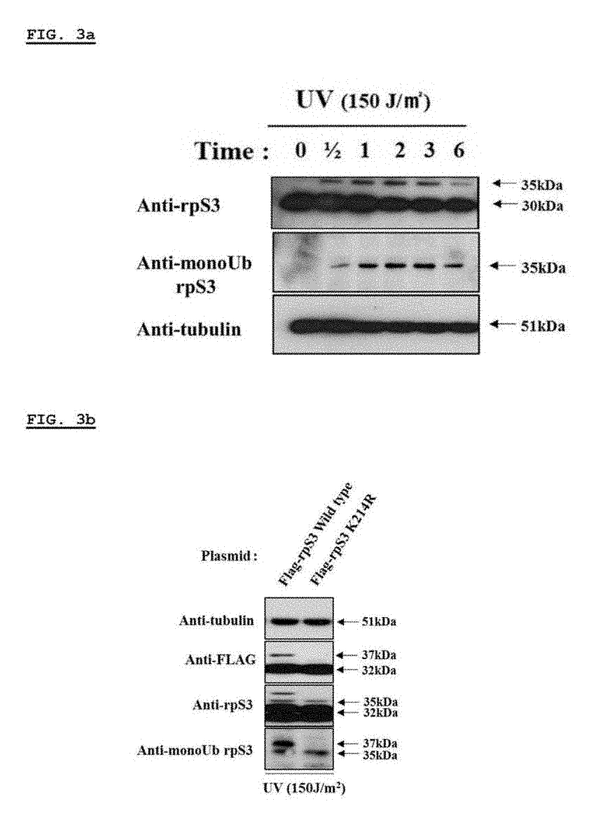

According to Western blotting results, when an antibody (anti-rpS3) recognizing the rpS3 protein was used, the signal indicated at the upper 35 kDa was detected simultaneously with the rpS3 protein (30 kDa). Next, in the case of using an antibody (anti-monoubiquitinated rpS3) recognizing the mono-ubiquitinated rpS3 obtained in Example 3, patterns the same as those of the upper 35 kD signal confirmed by the above antibody were indicated (FIG. 3a).

In view of an experimental technique, because a transfection experiment using plasmid expressing a mutant K214R rpS3-Flag fusion protein obtained by substituting the lysine 214 with arginine is easier than a replacement of all the rpS3 214 residues of the rpS3 gene in the cell, the following experiment was carried out. The HT1080 cell line was transfected after preparing a Flag-rpS3 wild type and a K214R mutant obtained by substituting the lysine 214 of rpS3 with arginine. After two days of the transfection, UV of 150 J/m² was irradiated and a confirmation is performed through the Western blotting (FIG. 3b).

First, according to a confirmation using the antibody against FLAG, it was found that the monoUb signal (37 kDa) was indicated only in the wild type because the exogenous Flag-rpS3 fusion protein has a Flag protein of 2 kDa. Next, it was found that, upon confirmation by the rpS3 antibody, the monoUb signal (35 kDa) of the endogenous rpS3 was indicated on both sides, however the monoUb signal of the exogenous Flag-rpS3 (37 kDa) was indicated only in the wild type. The above patterns appeared as same as those of the rpS3 monoUb antibody prepared by the inventors of the present invention.

Accordingly, it was confirmed that the rpS3 monoUb antibody prepared by the inventors of the present invention detects only the mono-ubiquitination specifically binding to the lysine 214 of the rpS3.

Then, the results after the human cell line HT1080 cell is subjected to various stresses such as Cycloheximide, Deoxynivalenol, Anisomycin, Blasticidin S, Pactamycin and Emetine were same as those after UV treatment.

### Example 5

Preparation of Yeast Antigen

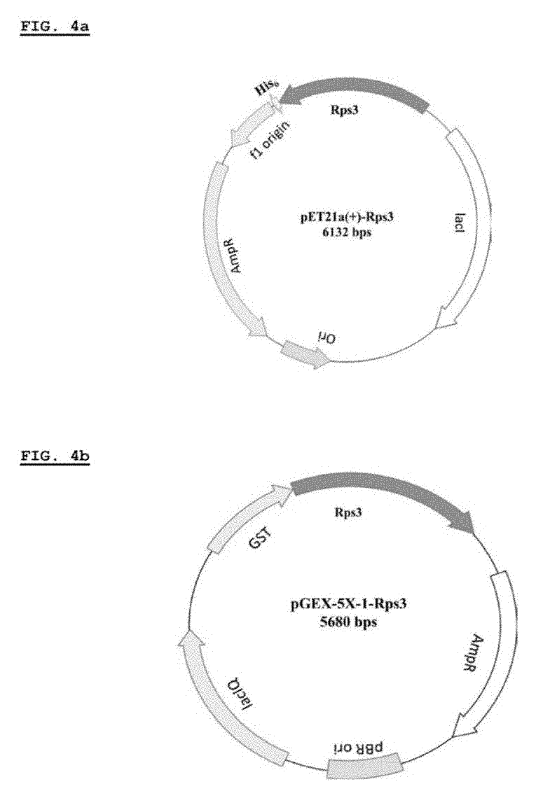

A BamHI-XhoI restriction enzyme site in a multiple cloning site (MCS) of pET-21a (Merck Millipore-Novagen Cat. #69740) is used, and whole protein coding gene (723 bp) of yeast Rps3p (Genbank accession number 271454 Y13139) was inserted, thus plasmid capable of expressing full-length Rps3 protein labeled with six histidine amino acids (6xHis) was established (FIG. 4a).

Recombinant bacteria was prepared by transforming the plasmid in E. coli-phylogenic protein expressing bacterial strain BL-21 (Amersham Pharmacia Biotech, Cat. #27-1542-01), and then the recombinant bacteria is cultured in lysogeny broth (LB) liquid medium for 3 hours at 37° C., strongly induced in a medium supplemented with 0.1 mM IPTG (Isopropyl β-D-1-thiogalactopyranoside), and cultured for 3 hours at 30° C. Cell walls and cell membranes of the above cultured bacteria were degraded by using a sonicator. The degraded bacteria is put into the nickel-NTA agarose resin having a property strongly binding to the 6xHis label, thereby enabling the 6xHis-labeled Rps3 protein to bind to the agarose resin through nickel ions.

Unspecific proteins were removed by washing the resin binding to the Rps3 protein with a wash solution of 20 times amounts of the resin, and fused proteins were collected from the resin by injecting elution buffer added therein with imidazole. After the imidazole was removed by the dialysis purification through a cellulose semipermeable membrane, the final concentration of the 6xHis-Rps3 fusion protein was 1 mg/mL.

### Example 6

Immunity

In order to prepare the polyclonal yeast Rps3 antibody, the 6xHis-Rps3 fusion protein obtained in Example 5 was intravenous-injected into a rabbit about 20 weeks old, and the immunity was reinforced through the second inoculation after two weeks from the first inoculation, and then the third inoculation after three weeks from the second inoculation.

### Example 7

Purification of Yeast Antibody

In order to purify the antibodies in the plasma collected from the rabbit after completion of the third immunization in Example 6 by using a humanitarian manner, the example was carried out as follows:

As shown in FIG. 4b, by using EcoRI and SalI in the MCS of a pGEX-5X-1 (Amersham Pharmacia Biotech Cat. #27-4584-01) vector, the coding gene of the Rps3 was fused, and plasmid pGEX-5X-1-Rps3 for expressing GST-Rps3 fusion recombination protein which is fused with GST protein was prepared. The plasmid was transformed in the BL-21 bacterial strain, the GST-rpS3 recombinant protein was expressed in the same manner as Example 5, cell lysate is put into GST-sepharose 4B resin (GE Healthcare Life Sciences, Cat. 17075601), and the GST-Rps3 protein was collected and purified.

The GST-Rps3 protein purified by the above manner was reacted with Affi-Gel 10 resin (Bio-Rad, Cat. #1536099) to prepare resin to which the GST-Rps3 is fixed, the reacted resin was passed through plasma collected from the immunized rabbit, and the antibody elution buffer (100 mM Glycine-HCl pH 2.4, 150 mM NaClm 4° C.) was reacted for 2 minutes, thus the antibodies specifically binding to the Rps3 protein were isolated.

### Example 8

Western Blotting Experiment on Yeast Using Polyclonal Antibody

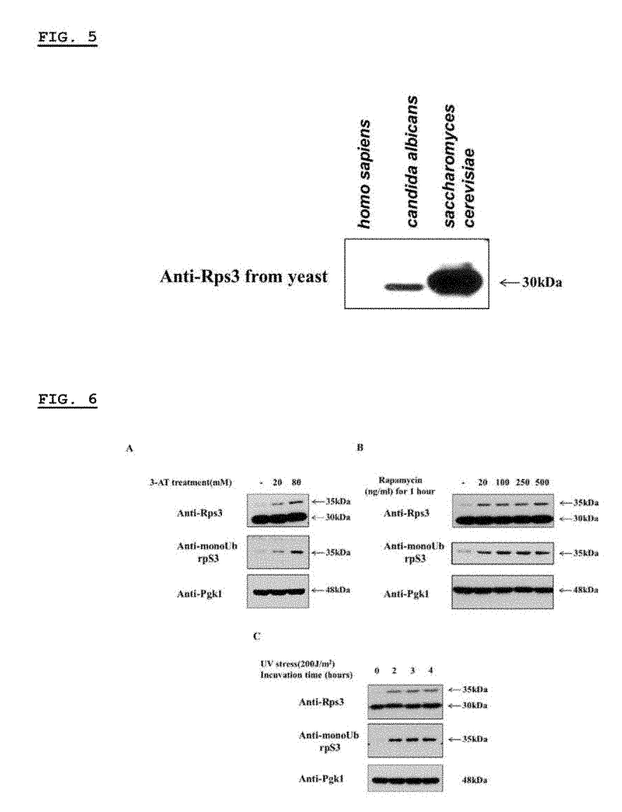

It was confirmed, through the Western blotting, whether human, Candida or yeast rpS3 protein is recognized by using the polyclonal antibody obtained in Example 7. The yeast antibody could not recognize the human protein, however, the antibody could recognize the Rps3 protein of the Candida or the yeast in the Western blotting (FIG. 5).

The yeast was harvested after receiving various stresses such as 3-AT (3-Amino-1,2,4-triazole), Rapamycin or ultraviolet rays, each cell of the yeast was put in glass beads with a Tris-Nacl-NP40 buffer solution (TAP lysis buffer) containing protease inhibitors, and the cell was lysed using a bead beater. The above obtained cell lysates were quantitated, and Western blotting experiment was performed by using 10 μg protein with respect to two kinds of antibodies.

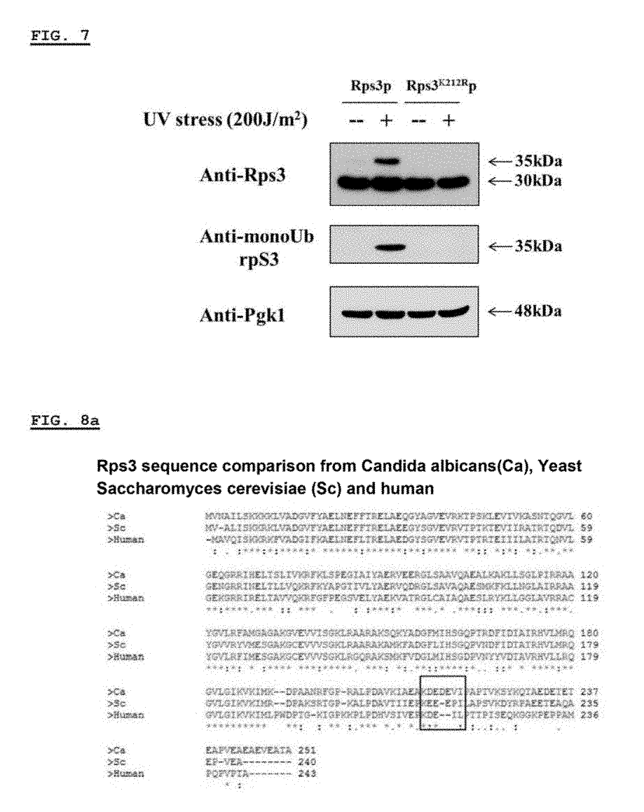

First, when the antibody recognizing the whole yeast Rps3 protein was used, the original rpS3 protein (30 kDa) and a signal (35 kDa) simultaneously indicated at an upper side thereof were detected (FIG. 6). Then, when the polyclonal antibody obtained in Example 7 was used, patterns the same as those of the upper signal 35 kDa confirmed by the above antibody were indicated (monoubiquitinated Rps3 in FIG. 3).

### Example 9

Experiment on Ubiquitin Binding of Yeast Rps3p Protein

Because the lysine 214 of the human rpS3 protein corresponds to lysine 212 in the yeast Rps3p protein, it was required to confirm whether the ubiquitin actually binds to the lysine 212 of the yeast Rps3p protein.

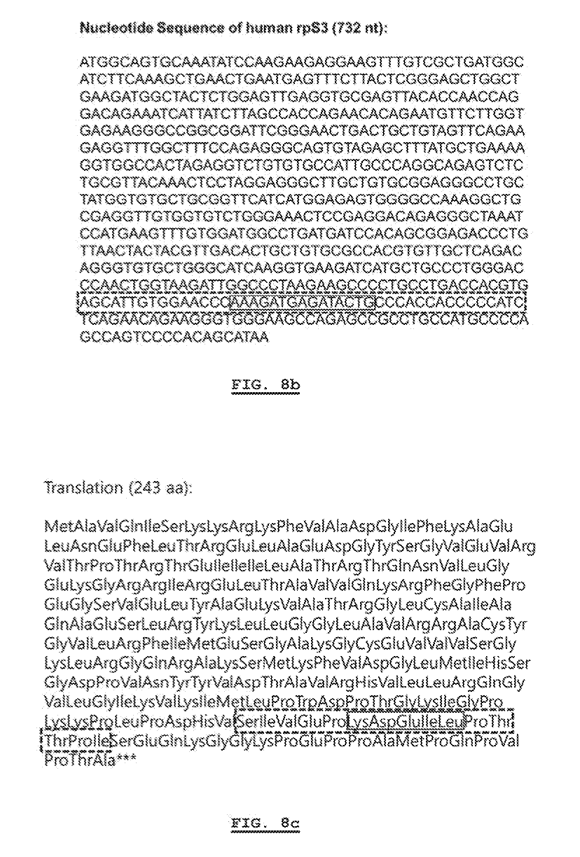

To this end, the lysine 212 of the yeast Rps3p protein was substituted with arginine, thus mutants which cannot bind to the ubiquitin in a living organism were prepared. According to the experimental result, it has been found that the Rps3 protein of the mutant yeast cell was not recognized as the polyclonal antibody in Example 7, and a band of 35 kDa was not indicated as mono-ubiquitin specific antibody (FIG. 7). It was found that the used amount of total protein was same, because the yeast Pgk1p used as a control group was indicated with the same amount in all conditions. Finally, it was found that the ubiquitin binds to the lysine 212 of the yeast Rps3p protein.

In the drawings the following abbreviations are used:

### GST: Glutathione-S-transferase

rpS3; ribosomal protein S3 (human)
pBR; name of the plasmid
lac IQ; lac repressor overproducing mutation due to the mutation of C-.>T at −0.35 of the promotor region of lad from E. coli
Lac I; lad gene from the lac operon that codes for the lac repressor
AmpR; Ampicillin resistant gene

What is claimed is:

1. An antibody prepared by using peptide having an amino acid structure of FIG. 1a as an antigen.

2. The antibody of claim 1, wherein the antibody specifically binds to rpS3 protein binding to ubiquitin protein.

3. A composition for diagnosing biological stress, the composition comprising an antibody which specifically binds to rpS3 protein binding to ubiquitin protein.

4. The composition of claim 3, wherein the antibody is prepared by using peptide having an amino acid structure of FIG. 1a as an antigen.

5. The composition of claim 3, wherein the antibody includes a monoclonal or polyclonal antibody.

6. A kit for diagnosing biological stress, the kit comprising the composition of claim 3.

7. A method of preparing peptide of FIG. 1a, the method comprising:
(a) synthesizing peptides (K-D-E-I-L) from lysine residue 214 to residue 218 of human rpS3 protein; and
(b) binding six residues (G-G-R-L-R-L) located at a carboxyl terminal of ubiquitin protein to an ε-amino group of lysine 214 of the peptide.

8. A method of preparing an antibody by using the peptide obtained by the method of claim 7 as an antigen.

9. A composition for diagnosing biological stress, the composition comprising an antibody prepared by using full-length yeast Rps3 protein labeled with a 6xHis tag as an antigen.


SEQUENCE LISTING (8 sequences)

SEQ ID NO: 1 - DNA, 15 bp, Homo sapiens
aaagatgagatactg

SEQ ID NO: 2 - DNA, 45 bp, Homo sapiens
agcattgtggaacccaaagatgagatactgcccaccacccccatc

SEQ ID NO: 3 - DNA, 18 bp, Homo sapiens
ctccgtctcagaggtggg

SEQ ID NO: 4 - DNA, 33 bp, Homo sapiens
accctgcacctggtgctccgtctcagaggtggg

SEQ ID NO: 5 - protein, 5 aa, Homo sapiens
KDEIL

SEQ ID NO: 6 - protein, 15 aa, Homo sapiens
SIVEPKDEILPTTPI

SEQ ID NO: 7 - protein, 6 aa, Homo sapiens
LRLRGG

SEQ ID NO: 8 - protein, 11 aa, Homo sapiens
TLHLVLRLRGG